(19) United States
(12) Patent Application Publication
(10) Pub. No.: US 2016/0237003 A1
(43) Pub. Date: Aug. 18, 2016

(54) METHOD FOR CONVERTING METHANE TO ETHYLENE

(71) Applicant: SAUDI BASIC INDUSTRIES CORPORATION, Riyadh (SA)

(72) Inventors: Aghaddin Kh. Mammadov, Houston, TX (US); Michael E. Huckman, Richmond, TX (US); Xiankuan Zhang, Houston, TX (US)

(21) Appl. No.: 15/028,986

(22) PCT Filed: Oct. 15, 2014
(86) PCT No.: PCT/US2014/060555

§ 371 Date: Apr. 13, 2016

Related U.S. Application Data
(60) Provisional application No. 61/891,452, filed on Oct. 16, 2013.

(51) Int. Cl. C07C 2/84; C07C 5/327

(52) U.S. Cl. CPC C07C 2/84; C07C 5/327; C07C 2521/08; C07C 2523/34

(57) ABSTRACT

In an embodiment, a process for producing ethylene comprising: introducing a methane stream comprising methane, oxygen, and water to a methane coupling zone; reacting the methane, the oxygen, and the water in the methane coupling zone via a methane oxidative coupling reaction to produce a first product stream; introducing the first product stream to a pyrolysis zone; and pyrolyzing ethane in the first product stream in the pyrolysis zone to produce a second product stream comprising ethylene. A heat from the methane coupling reaction is used in the pyrolysis reaction.

### TECHNICAL FIELD

The present disclosure relates generally to systems and processes for converting hydrocarbon-containing feed streams to olefins and other products, and more particularly to systems and processes for converting natural gas to ethylene and other products.

### BACKGROUND

Oxidative coupling of methane conversion for ethylene production has been previously described. This technology has experienced limited industrial realization, however, due to the presence of issues such as low yield and low concentration of ethylene (C₂H₄) in the products. Such issues can lead to high separation costs.

Prior attempts at oxidative coupling of methane conversion for ethylene production have included the use of basic oxide catalysts and mixtures for oxidative conversion of methane to C₂₊ hydrocarbons. The products of oxidative conversion of methane typically include ethylene (C₂H₄), ethane (C₂H₆), carbon monoxide (CO), and carbon dioxide (CO₂). At high concentrations of the target product, ethylene, consecutive deep oxidation of ethylene to CO and CO₂ can occur, where deep oxidation refers to consecutive deep oxidation of ethane and ethylene to CO and CO₂. Variations of the reaction conditions during the reaction often cause the concentration of ethylene in the outlet gas to be less than 8 volume percent (vol %). Consequently, separation costs for the ethylene from the outlet mixture of products is often expensive and causes limitations for realization of these conversion processes.

There accordingly remains a need for more efficient and improved systems and processes for the conversion of natural gas and other feed streams to olefins, namely to ethylene, with sufficient efficiency to make the process commercially viable.

### BRIEF SUMMARY

Disclosed herein is a process for converting methane to olefins.

In an embodiment, a process for producing ethylene comprising: introducing a methane stream comprising methane, oxygen, and water to a methane coupling zone; reacting the methane, the oxygen, and the water in the methane coupling zone via a methane oxidative coupling reaction to produce a first product stream; introducing the first product stream to a pyrolysis zone; and pyrolyzing ethane in the first product stream in the pyrolysis zone to produce a second product stream comprising ethylene. A heat from the methane coupling reaction is used in the pyrolysis reaction.

The above described and other features are exemplified by the following figures and detailed description.

### BRIEF DESCRIPTION OF THE DRAWINGS

Refer now to the figures, which are exemplary embodiments, and wherein the like elements are numbered alike.

FIG. 1 illustrates a dual reactor process and system for the production of olefins.

FIG. 2 illustrates a single reactor process and system for the production of olefins.

### DETAILED DESCRIPTION

Disclosed herein is a system and process that provides for conversion of hydrocarbon-containing feed streams to olefins, e.g., the conversion of natural gas to ethylene. The process uses the integration of the methane oxidative conversion (also referred to herein as a methane coupling reaction or as catalytic oxidative conversion) reaction of a feed stream comprising methane (such as methane separated from natural gas) with the process of steam cracking of ethane and optionally other C₃₊ hydrocarbons. The energy for the endothermic pyrolysis reaction of ethane and optionally other hydrocarbons is provided for by the oxidative conversion of methane. In other words, the heat of the methane oxidative conversion reaction can be used for hydrocarbon cracking, for example, of ethane. The system and process can also result in the conversion of unconverted methane from the oxidative conversion through radical intermediates of the cracking process to result in an increase of the total concentration of ethylene in the products. Used herein, C₂₊ hydrocarbons are hydrocarbons with two or more carbon atoms, for example, 2 to 4, for example, 2 to 3 and C₃₊ hydrocarbons are hydrocarbons with three or more carbon atoms, for example, 3 to 4.

The integrated technology disclosed herein utilizes the exothermic catalytic methane oxidative conversion process in conjunction with the endothermic pyrolysis process. The system and process disclosed herein also allows for ethane from the methane oxidative conversion reactor to optionally be combined with additional hydrocarbons (such as additional ethane, propane, butane, naphtha, or a combination comprising one or more of the foregoing) in the pyrolysis reactor.

Through integration of methane oxidative conversion with the process of cracking of hydrocarbons, simultaneous production of ethylene from natural gas and from cracking of hydrocarbons can be achieved. Therefore cracking of hydrocarbons such as naphtha takes place using the heat of the exothermic reaction of methane oxidative coupling. As used herein, “simultaneous production of ethylene” refers to the formation of ethylene both from methane and ethane or from methane and other C₂₊ hydrocarbons (such as naphtha) using the integrated processes disclosed herein. Accordingly, it is expected that olefin yield and C₂H₄ concentration in the products can be increased.

The catalyst for use in the methane oxidative conversion process can be one or more metal oxides, for example, a mixture of two metal oxides. The metal oxide can comprise an oxide of Li, Mg, Sr, La, Na, Mn, or a combination comprising one or more of the foregoing. When used as a metal oxide mixture, exemplary combinations are Na—Mn—O, Li—Mg—O, and Sr—La—O, where, for example Na—Mn—O is a mixture of sodium oxide and manganese oxide, Li—Mg—O is a mixture of lithium oxide and magnesium oxide, and Sr—La—O is a mixture of strontium oxide and lanthanum oxide. For example, and while not to be construed as limiting, the catalyst can be a Na—Mn—O mixture, which is a mixture of Na oxide and Mn oxides. Such catalysts can also include preparation using inert support, such as silica, Al₂O₃, MgO, or the like. For example, the catalyst can be Na—Mn—O/SiO₂, where the catalyst can be prepared through impregnation of SiO₂ with NaOH and Mn(CH₃COO)₂, followed by drying at 120 degrees Celsius (° C.) for 12 hours, and then calcination at 750° C. The Na—Mn—O/SiO₂ catalyst can contain 3 to 15 weight percent (wt %) Na and 5 to 25 wt % Mn based on the total weight of the catalyst and support.

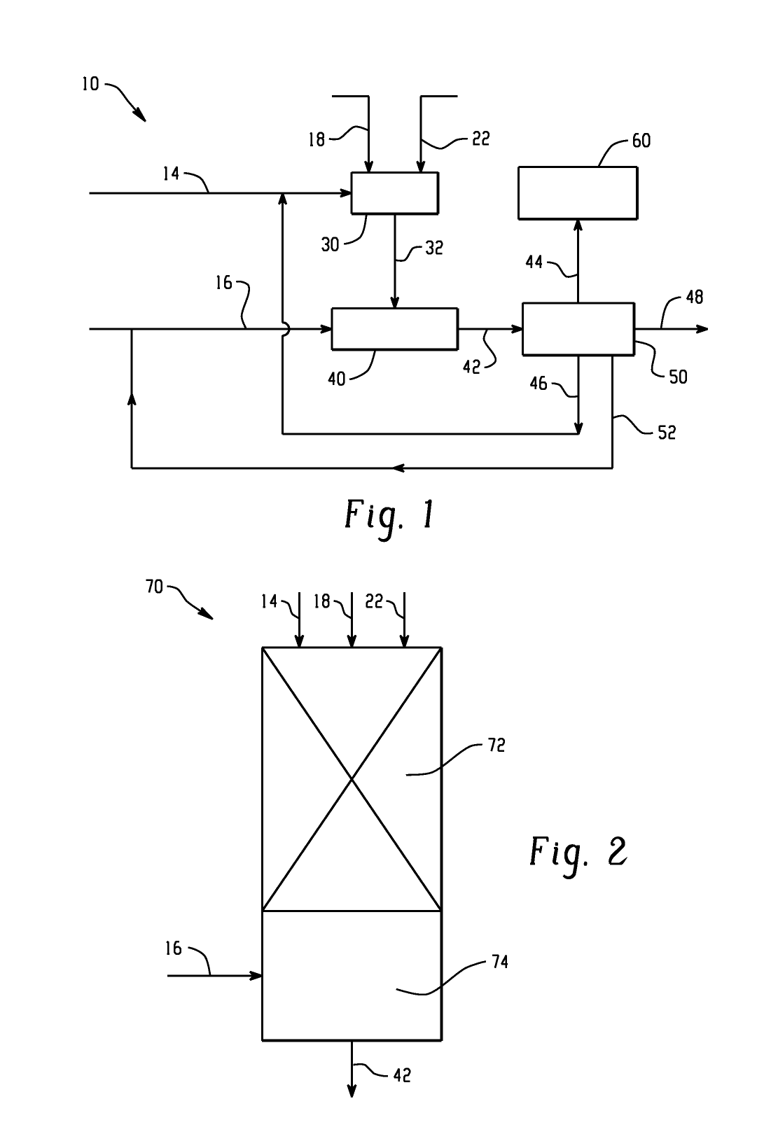

Referring now to the figures, FIG. 1 illustrates an exemplary process and system for the production of olefins and for example, showing the production of chemicals from natural gas and the integration of an oxidative coupling reactor with a pyrolysis reactor.

FIG. 1 shows system 10 that includes oxidative conversion reactor 30, pyrolysis reactor 40, separation unit 50, and power plant 60. It is noted that methane product stream 44 produced using the systems and processes disclosed herein can be used in applications other than power plant 60.

FIG. 1 shows methane stream 14 enters oxidative conversion reactor 30. Methane stream 14 can comprise methane separated from natural gas, where natural gas generally comprises greater than or equal to 85 vol %, for example, 85 to 90 vol % methane and less than 15 vol %, for example, 10 to 15 vol % ethane based on the total volume of the natural gas. The separation of the natural gas can occur, for example, using a cold box. The methane stream 14 can comprise, greater than or equal to 85 vol %, for example, greater than or equal to 90 vol % of the total methane in the natural gas.

Methane stream 14 can be combined with recycle methane feed stream 46, either in the reactor 30, or upstream of the oxidative conversion reactor 30. Oxygen source 18 and water source 22 are also supplied to oxidative conversion reactor 30. Oxygen source 18 can, for example, be pure oxygen or air. The volume ratio of methane to oxygen to water used as feed to methane oxidative conversion reactor 30 can be 2-7:1:2-3, for example, 2-3.5:1:3. The volume ratio of methane to oxygen (CH₄/O₂ ratio) can be 2 to 6. Such ranges can allow for control of reaction conditions in oxidative conversion reactor 30 and can allow the formation of 3 to 7 vol % ethylene.

Methane, oxygen (which can be oxygen in air), and water are reacted in methane oxidative reactor 30 to provide methane oxidative reaction product stream 32. The methane oxidative conversion reaction can occur at a temperature of 700 to 900° C., for example, 750 to 850° C., for example, 800 to 850° C. The methane oxidative conversion reaction can occur at a pressure of 0 to 20 bar. The space velocity can be 3600 to 36000 1/hour, for example, 3600 to 7200 1/hour and can have a contact time of 0.1 to 1 second. As discussed above, a catalyst (such as Na—Mn—O/SiO₂) can be used in oxidative conversion reactor 30.

Product stream 32 can comprise C₂H₄, C₂H₆, CO₂, CO₂, CH₄, water, or a combination comprising one or more of the foregoing. Product stream 32 can be free of oxygen, where the stream can comprise less than or equal to 0.5 vol %, for example, 0 vol % oxygen. The ratio of the components of the reaction can be selected to realize high selectivity of the reaction (e.g. 70 to 75%) with a level of conversion, which leads to the formation of 3 to 7 vol %, for example 3 to 4 vol % of ethylene. If air is used as a feed stream to oxidative conversion reactor 30 rather than oxygen, product stream 32 can also contain nitrogen.

Product stream 32 is then fed to pyrolysis reactor 40. Product stream 32 can be fed to pyrolysis reactor 40 without separation of the water. The heat from the methane oxidation reaction can be transferred directly by the product stream 32 to the pyrolysis zone or can be carried indirectly, for example, by a heating fluid that is heated up in the presence of the methane oxidation reaction (for example as a heating jacket surrounding the reactor or as separate channels flowing through the reaction zone) and is transferred to the pyrolysis zone (for example as a heating jacket surrounding the reactor or as separate channels flowing through the reaction zone).

Hydrocarbon stream 16 can also optionally be added to pyrolysis reactor 40. Likewise, hydrocarbon stream 16 can be first combined with product stream 32 upstream of the pyrolysis reactor. Hydrocarbon stream 16 can comprise C₂₊ hydrocarbons, for example, C₂₋₄ hydrocarbons. For example, hydrocarbon stream 16 can comprise ethane (such as ethane that has been separated from natural gas), propane, butane, naphtha or a combination comprising one or more of the foregoing. Optionally, steam can be added to hydrocarbon stream 16, where hydrocarbon stream 16 can comprise 40 to 50 vol % steam based on the total volume of the stream. Likewise, steam can be added to pyrolysis reactor 40 as a separate stream such that the hydrocarbons entering the reactor are diluted to 50 to 60 vol % based on the hydrocarbon stream 16 and the steam stream. Hydrocarbon stream 16 can contain C₃₊ hydrocarbons in an amount of 0 to 5 vol % based on the total volume of the hydrocarbons in the stream. Likewise, hydrocarbon stream 16 can comprise naphtha. It is understood that while methane stream 14 and hydrocarbon stream 16 can originate from natural gas, alternative sources for methane and ethane can be utilized.

The total volume ratio of methane to ethane fed into pyrolysis reactor 40, for example from product stream 32 and, where present, hydrocarbon stream 16 and recycle ethane stream 52 can be 1-12:1, for example, 5-10:1. For example, the ratio of CH₄:C₂H₆ can be 8 to 12 by volume (e.g., 10).

The reaction in pyrolysis reactor 40 can occur at a temperature of 750 to 900° C., for example, 840 to 860° C. (e.g., 850° C.). The reaction in pyrolysis reactor 40 can occur at a temperature of 790 to 810° C. (e.g., 800° C.). The temperature can depend on the temperature and components of a hydrocarbon feed. For example, if hydrocarbon stream 16 comprises ethane, the reaction temperature in pyrolysis reactor 40 can be 790 to 810° C. (e.g. 800° C.), whereas if hydrocarbon stream 16 comprises naphtha, the reaction temperature can be 840 to 860° C. (860° C.). The pyrolysis reactor can be free of a catalyst, for example, it can have no added catalyst.

Heat in product stream 32 from the exothermic methane oxidative conversion reaction is transferred to pyrolysis reactor 40 for the endothermic reaction therein. Because product stream 32 can be added without separation to pyrolysis reactor 40, the heat of the exothermic methane oxidative conversion can be directly applied (without cooling) to the endothermic cracking reaction (also referred to herein as a dehydrogenation reaction). The physical energy required for the ethane cracking thus consumes the heat applied by the methane exothermic oxidative conversion. Accordingly, the heat of the methane oxidative reaction is generally not used for utility purpose such as for generation of heat during cooling. Rather the heat of the methane oxidative conversion reaction can be directly applied to the endothermic reaction.

It is noted that a first portion of the heat and/or product stream 32 can be used for other purposes than as feed to pyrolysis reactor 40 while a second portion of the heat and/or products in product stream 32 can be introduced into pyrolysis reactor 40.

Pyrolysis product stream 42 can comprise C₄H₈, C₃H₆, C₂H₄, C₂H₆, CO₂, CO, CH₄, H₂O, or a combination comprising one or more of the foregoing. If air is used as a feed stream to methane oxidative conversion reactor 30 rather than oxygen, pyrolysis product stream 42 can also contain nitrogen. Pyrolysis product stream 42 can comprise greater than or equal to 10 vol % ethylene based on the total volume of the product stream.

Pyrolysis product stream 42 exits pyrolysis reactor 40 and can be introduced to separation unit 50. Separation unit 50 can comprise one or more separation units. Separation unit 50 can be, for example, a cold box that performs a cryogenic separation. Separation unit 50 produces product ethylene stream 48, ethane stream 52, methane product stream 44, recycle methane feed stream 46, or a combination comprising one or more of the foregoing.

FIG. 1 shows that methane product stream 44 can be used for fuel in power plant 60 for production of energy. In addition or alternatively, methane product stream 44 can be used in other applications such as combustion fuel for heat in endothermic reactions, such as methane steam reforming processes that produce syngas (a gaseous mixture containing hydrogen (H₂) and carbon monoxide (CO), which may further contain other gas components like carbon dioxide (CO₂), water (H₂O), methane (CH₄), nitrogen (N₂), or a combination comprising one or more of the foregoing). All or a portion of methane product stream 44 can be used for fuel, for example, when nitrogen is present. In addition to methane product stream 44, methane can alternatively or additionally be separated as recycle methane stream 46.

As shown in FIG. 1, recycle methane stream 46 can be combined with methane stream 14 for feed to methane oxidative conversion reactor unit 30. Alternatively, recycle methane stream 46 can be used as a separate methane feed (alone or in conjunction with methane feed stream 14) to methane oxidative conversion reactor 30. As further shown in FIG. 1, recycle ethane stream 52 can be recycled and combined with hydrocarbon stream 16 as additional pyrolysis feed to pyrolysis reactor 40. Recycle ethane stream 52 can likewise be fed directly into pyrolysis reactor 40.

FIG. 2 illustrates that the methane oxidative conversion reaction and the pyrolysis reaction can be conducted in one reactor. As shown, such processes can be carried out using reactor 70. Reactor 70 (e.g. reactor tube) can be any suitable reactor that comprises two separate zones: methane coupling zone 72 and pyrolysis zone 74. Feed to methane coupling zone 72 in reactor 70 can include methane stream 14, oxygen source 18, and water source 22. As discussed hereinabove, natural gas can be separated to provide methane stream 14 and hydrocarbon stream 16. The reaction conditions and volume ratios of the feed into the methane coupling zone 72 can be the same as those described above for the oxidative conversion reactor 30. Following reaction in the methane coupling zone 72, products and heat from methane coupling zone 72 can be combined with optional hydrocarbon stream 16 in pyrolysis zone 74. The reaction conditions and volume ratios of the feed into the pyrolysis zone 74 can be the same as those described above for the pyrolysis reactor 40.

Pyrolysis product stream 42 exiting reactor 70 can be further processed as described hereinabove with reference to FIG. 1. For example, pyrolysis product stream 42 exiting reactor 70 can be subjected to separation in separation unit 50 for example by cryogenic separation in a cold box. Separation unit 50 allows for production of product ethylene stream 48, ethane stream 52, methane product streams 44, and/or recycle methane stream 46 (shown in FIG. 1). When air is used as oxygen source 18 rather than oxygen, nitrogen can also be separated and removed to the atmosphere.

The following examples are provided to illustrate various aspects of the present invention. The examples are merely illustrative and are not intended to limit the systems and processes made in accordance with the disclosure of the materials, conditions, or process parameters set forth therein.

### EXAMPLES

### Example 1

In this Example, methane oxidative conversion was carried out using Na—Mn—O/SiO₂ as a catalyst. Ethane, together with the products of the first reaction, was used as pyrolysis feeds to the second reactor.

The catalyst, Na—Mn—O/SiO₂ was prepared through impregnation of SiO₂ with NaOH and Mn(CH₃COO)₂, which was then dried at 120° C. for 12 hours, and then calcined at 750° C. The content of Na and Mn in the catalyst was 8% and 15 wt %, respectively. 2.5 g of the above-mentioned catalyst were contained in the methane oxidative conversion reactor. The flow rates to the methane oxidative reactor were: 100 cubic centimeters per minute (cc/min) CH₄, 30 cc/min O₂, and 7.2 milligrams per minute (mg/min) water. The reaction temperature was 850° C. and the volume ratio of the reaction components was CH₄:O₂:H₂O=3.3:1:3.

The concentration of the components of the gas mixture and the results of the reaction in the methane oxidative reactor were as follows:

| | |
|---|---|
| CH₄ conversion, mole % | 28.0 |
| Selectivity to C₂₊, mole % | 70.0 |
| C₂₊ yield, mole % | 19.6 |
| C₂H₄ concentration in outlet gas, mole % | 7.2 |
| C₂H₆ concentration in outlet gas, mole % | 3.6 |
| CO₂ + CO concentration in the outlet gas, mole % | 9.3 |
| CH₄ concentration in the outlet gas, mole % | 79.8 |

The output of the first reaction from the methane oxidative conversion reactor containing the above-mentioned products was fed to the second reactor, where 10 cc/min of ethane was added. The temperature in the pyrolysis reactor was maintained at 800° C. The volume ratio of methane and ethane used as feed to the pyrolysis reactor was CH₄/C₂H₆=10 by volume. The products from the pyrolysis reactor were cooled to room temperature. After the reactor was cooled, the gas was separated from water and then was fed to the gas chromatographer (GC).

The output of the reaction from the second reactor had a composition as follows:

| | |
|---|---|
| C₂H₄ concentration in outlet gas, mole % | 12.3 |
| C₂H₆ concentration in outlet gas, mole % | 5.1 |
| CO₂ + CO concentration in the outlet gas, mole % | 9.2 |
| CH₄ concentration in the outlet gas, mole % | 73.3 |

After separation of ethylene, CO, and CO₂ from the product stream, methane could have been recycled to the methane oxidative conversion reactor and/or used as fuel for a power plant or the like, while the ethane is fed to the secondary pyrolysis reactor.

### Example 2

The experiments of Example 2 were carried out as in Example 1, except that air rather than oxygen was used for the methane oxidative coupling reaction.

2.5 g of the above-mentioned catalyst was contained in the methane oxidative conversion reactor. The flow rates to the methane oxidative reactor were: 100 cc/min CH₄, 150 cc/min air, and 7.2 mg/min water. The reaction temperature was 850° C.

The distribution of the final products after the methane oxidative reactor was as follows:

| | |
|---|---|
| CH₄ conversion, mole % | 30.0 |
| Selectivity to C₂₊, mole % | 68.0 |
| C₂₊ yield, mole % | 20.4 |
| C₂H₄ concentration in outlet gas, mole % | 3.2 |
| C₂H₆ concentration in outlet gas, mole % | 1.6 |
| CO₂ + CO concentration in the outlet gas, mole % | 4.5 |
| CH₄ concentration in the outlet gas, mole % | 33.1 |

It is observed that while the conversion of CH₄ in Example 2 (30.0 mole %) was similar to Example 1 (28.0 mole %), the CH₄ concentration in the outlet gas in Example 2 was much less (33.1 mole %) than that of Example 1 (79.8 mole %). Without being bound by theory, this reduction in methane concentration is believed to be due to the use of air rather than oxygen, which led to the dilution of the reaction components and products with nitrogen. In addition, C₂H₄ concentration in the outlet dry gas in Example 2 (3.2 mole %) was less than in Example 1 (7.2 mole %) and C₂H₆ concentration in the outlet dry gas in Example 2 (1.6 mole %) was less than in Example 1 (3.6 mole %). This difference is also believed to be due to the dilution of the components of the gas mixture by nitrogen.

The output from the first reactor (i.e., from the methane oxidative conversion reactor) containing the above-mentioned products, including the remaining methane, was fed to the second reactor without cooling and without separation of water, where 15 cc/min ethane was fed by a separate line. The temperature in the second reactor was maintained at 850° C. The volume ratio of methane and ethane used as feed was CH₄/C₂H₆=6.6.

The output from the second reactor (i.e. the pyrolysis reactor) had a composition as follows:

| | |
|---|---|
| C₂H₄ concentration in outlet gas, mole % | 9.6 |
| C₂H₆ concentration in outlet gas, mole % | 3.7 |
| CO₂ + CO concentration in the outlet gas, mole % | 5.2 |
| CH₄ concentration in the outlet gas, mole % | 28.3 |
| N₂ concentration in outlet gas, mole % | 54.0 |

In this example, the methane, diluted with nitrogen, can be fed to a power plant for generation of electricity. Following separation, the ethane can be recycled to the pyrolysis reactor.

Without being bound by theory, it is believed that the CH₄ concentration in the outlet gas from the pyrolysis reactor in Example 2 (28.3 mole %) was much less than in Example 1 (73.3 mole %) due to the presence of the nitrogen in the output in Example 2.

### Example 3

The experiments of Example 3 were carried out as in Example 1, except that methane and ethane were fed to the same reactor in the form of a distributed feed such as one similar to that discussed above with reference to FIG. 2. Methane was fed to the catalyst zone and ethane was fed to the empty zone following the catalyst zone, where it was mixed with the gas delivered from the catalyst zone. In this example, the second reactor, which was used as a thermal reactor without catalyst, was located after the catalyst zone of the same reactor and the methane oxidative conversion reactor was utilized without the use of a separate secondary pyrolysis reactor (i.e. different zones in the same reactor are utilized).

2.5 g of the above-mentioned catalyst was contained in the methane oxidative conversion reactor or catalyst zone. The flow rates to the reactor were: 100 cc/min CH₄, 150 cc/min air, and 7.2 mg/min water to the catalyst zone and 15 cc/min C₂H₆ was added to the second zone of the reactor. The volume ratio of methane and ethane used as feed in the second zone was CH₄/C₂H₆=6.6.

Distribution of the final products after the methane oxidative reactor (without water) was as follows:

| Temperature of the methane oxidative conversion reaction: | 850° C. |
|---|---|
| CH₄ conversion, mole % | 23.0 |
| C₂H₄ concentration in outlet gas, mole % | 7.2 |
| C₂H₆ concentration in outlet gas, mole % | 1.2 |
| CO₂ + CO concentration in the outlet gas, mole % | 7.4 |
| CH₄ concentration in the outlet gas, mole % | 33.7 |
| N₂ concentration in outlet gas, mole % | 52.6 |

C₂H₄ selectivity was not calculated due to the formation of ethylene from methane and ethane.

The concentration of ethylene produced in Example 3 was less than the concentration of ethylene produced in Example 2, while CO and CO₂ produced in Example 3 was more than the amount of CO and CO₂ produced in Example 2. These examples show that processing can be successful using one or two reactors.

### Example 4

The experiments in Example 4 were carried out as in Example 1, except that 57.5 mg/min naphtha was fed to the secondary pyrolysis reactor rather than ethane.

2.5 g of the above-mentioned catalyst was contained in the methane oxidative conversion reactor. The flow rates to the methane oxidative reactor were: 100 cc/min CH₄, 150 cc/min air, and 7.2 mg/min water.

Distribution of the final products after the reactor was as follows:

| Temperature of the methane oxidative conversion reaction: | 850° C. |
|---|---|
| CH₄ conversion, mole % | 30.0 |
| Selectivity to C₂₊, mole % | 68.0 |
| C₂₊ yield, mole % | 20.4 |
| C₂H₄ concentration in outlet gas, mole % | 3.2 |
| C₂H₆ concentration in outlet gas, mole % | 1.6 |
| CO₂ + CO concentration in the outlet gas, mole % | 4.5 |
| CH₄ concentration in the outlet gas, mole % | 33.1 |

The output from the second reactor (i.e. the pyrolysis reactor) had a composition as follows:

| | |
|---|---|
| C₂H₄ concentration in outlet gas, mole % | 8.0 |
| C₂H₆ concentration in outlet gas, mole % | 1.3 |
| C₃H₆ concentration in outlet gas, mole % | 4.5 |
| C4 hydrocarbons concentration in the outlet gas, mole % | 1.5 |
| CO₂ + CO concentration in the outlet gas, mole % | 7.8 |
| CH₄ concentration in the outlet gas, mole % | 32.2 |
| H₂ concentration in the outlet gas, mole % | 2.8 |
| N₂ concentration in outlet gas, mole % | 42.1 |

While the C₂H₄ concentration in Example 4 after the pyrolysis reaction was 8 mol % and the C₂H₄ in Example 1 after the pyrolysis reaction was 12.3 mol %, Example 4 in oxidative coupling step produced less ethylene than in Example 1 due to the presence of air instead of oxygen. Thus, the integration of methane oxidative conversion with naphtha cracking led to increased ethylene concentration, even in the presence of air. Without being bound by theory, this increase is believed to be due to the formation of more ethylene molecules from one mole of naphtha than that of one mole of ethane.

The present disclosure provides a process for conversion of natural gas to olefins, for example, to ethylene by integration of the process of catalytic natural gas oxidative conversion with the cracking of the C₂₊ hydrocarbons, wherein the energy for endothermic pyrolisis of C₂₊ hydrocarbons is provided by catalytic conversion of methane. The process allows for the use of heat from the methane oxidative conversion process for cracking of C₂₊ hydrocarbons to realize the secondary conversion of unconverted methane by the radical intermediates of the cracking process and to increase total concentration of ethylene in the products, making easy separation of olefin from the reaction components.

The present disclosure comprises the option that the oxidation and pyrolysis reactor is combined in one reactor consisting of two zones, where a first zone of the reactor is used for oxidative conversion of methane and a second catalytic zone of the reactor is used for the cracking of hydrocarbons using the heat of the exothermic oxidative conversion reaction.

Set forth below are some embodiments of the present catalyst, and methods of making and using the catalyst.

### Embodiment 1

a process for producing ethylene comprising: introducing a methane stream comprising methane, oxygen, and water to a methane coupling zone; reacting the methane, the oxygen, and the water in the methane coupling zone via a methane oxidative coupling reaction to produce a first product stream; introducing the first product stream to a pyrolysis zone; and pyrolyzing ethane in the first product stream in the pyrolysis zone to produce a second product stream comprising ethylene. Heat from the methane coupling reaction is used in the pyrolysis reaction.

### Embodiment 2

The process of Embodiment 1, wherein the second product stream comprises greater than or equal to 10 vol % ethylene based on the total volume of the second product stream.

### Embodiment 3

The process of any of Embodiments 1-2, wherein the methane oxidative coupling reaction is facilitated by a catalyst and wherein the catalyst comprises a metal oxide, wherein the metal comprises Li, Mg, Sr, La, Na, Mn, or a combination comprising one or more of the foregoing.

### Embodiment 4

The process of Embodiment 3, wherein the catalyst comprises Na—Mn—O/SiO₂.

### Embodiment 5

The process of any of Embodiments 1-4, further comprising separating natural gas into the methane stream and an ethane stream prior to introducing the methane stream.

### Embodiment 6

The process of Embodiment 5, further comprising introducing the ethane stream to the pyrolysis zone.

### Embodiment 7

The process of any of Embodiments 1-6, wherein the oxygen stream comprises air.

### Embodiment 8

The process of any of Embodiments 1-7, further comprising separating a recycle ethane stream from the second product stream and introducing the recycle ethane stream to the pyrolysis zone.

### Embodiment 9

The process of any of Embodiments 1-7, wherein the second product stream comprises methane and the methane is directed to a power plant and used as a fuel source.

### Embodiment 10

The process of any of Embodiments 1-9, wherein all of the first product stream is introduced into the pyrolysis zone.

### Embodiment 11

The Process of any of Embodiments 1-10, Wherein the methane oxidative coupling reaction occurs in an oxidative conversion reactor and the pyrolyzing occurs in a pyrolysis reactor.

### Embodiment 12

The process of any of Embodiments 1-10, wherein the methane oxidative coupling reaction occurs in an oxidative conversion zone in a reactor and the pyrolyzing occurs in a pyrolysis zone in the same reactor.

### Embodiment 13

The process of any of Embodiments 1-12, further comprising introducing a hydrocarbon stream to the pyrolysis zone.

### Embodiment 14

The process of Embodiment 13, wherein the hydrocarbon stream comprises naphtha.

### Embodiment 15

The process of Embodiment 13, wherein the hydrocarbon stream comprises ethane.

### Embodiment 16

The process of any of Embodiments 1-13, wherein the coupling reaction occurs at a temperature of 750 to 900° C.

### Embodiment 17

The process of Embodiment 16, wherein the reaction occurs at a temperature of 790 to 810° C.

### Embodiment 18

The process of any of Embodiments 1-17, further comprising separating a purified ethylene from the second product stream.

In general, the invention may alternatively comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

All ranges disclosed herein are inclusive of the endpoints, and the endpoints are independently combinable with each other (e.g., ranges of “up to 25 wt %, or, for example, 5 to 20 wt %”, is inclusive of the endpoints and all intermediate values of the ranges of “5 to 25 wt %,” etc.). “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the film(s) includes one or more films). Reference throughout the specification to “one embodiment,” “another embodiment,” “an embodiment,” and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments. This application claims priority to U.S. Patent Application 61/891,456 both filed Oct. 16, 2013, which is incorporated herein by reference in its entirety.

While particular embodiments have been described, alternatives, modifications, variations, improvements, and substantial equivalents that are or may be presently unforeseen may arise to Applicants or others skilled in the art. Accordingly, the appended claims as filed and as they may be amended are intended to embrace all such alternatives, modifications variations, improvements, and substantial equivalents.

Disclosure of a narrower range or more specific group in addition to a broader range is not a disclaimer of the broader range or larger group.

I/We claim:

1. A process for producing ethylene comprising:
introducing a methane stream comprising methane, oxygen, and water to a methane coupling zone;
reacting the methane, the oxygen, and the water in the methane coupling zone via a methane oxidative coupling reaction to produce a first product stream;
introducing the first product stream to a pyrolysis zone; and
pyrolyzing ethane in the first product stream in the pyrolysis zone to produce a second product stream comprising ethylene;
wherein a heat from the methane coupling reaction is used in the pyrolysis reaction.

2. The process of claim 1, wherein the second product stream comprises greater than or equal to 10 vol % ethylene based on the total volume of the second product stream.

3. The process of claim 1, wherein the methane oxidative coupling reaction is facilitated by a catalyst and wherein the catalyst comprises a metal oxide, wherein the metal comprises Li, Mg, Sr, La, Na, Mn, or a combination comprising one or more of the foregoing.

4. The process of claim 3, wherein the catalyst comprises Na—Mn—O/SiO₂.

5. The process of claim 1, further comprising separating natural gas into the methane stream and an ethane stream prior to introducing the methane stream.

6. The process of claim 5, further comprising introducing the ethane stream to the pyrolysis zone.

7. The process of claim 1, wherein the oxygen stream comprises air.

8. The process of claim 1, further comprising separating a recycle ethane stream from the second product stream and introducing the recycle ethane stream to the pyrolysis zone.

9. The process of claim 1, wherein the second product stream comprises methane and the methane is directed to a power plant and used as a fuel source.

10. The process of claim 1, wherein all of the first product stream is introduced into the pyrolysis zone.

11. The process of claim 1, wherein the methane oxidative coupling reaction occurs in an oxidative conversion reactor and the pyrolyzing occurs in a pyrolysis reactor.

12. A process for producing ethylene comprising:
introducing a methane stream comprising methane, oxygen, and water to a methane coupling zone;
reacting the methane, the oxygen, and the water in the methane coupling zone via a methane oxidative coupling reaction to produce a first product stream;
introducing the first product stream to a pyrolysis zone; and
pyrolyzing ethane in the first product stream in the pyrolysis zone to produce a second product stream comprising ethylene;
wherein a heat from the methane coupling reaction is used in the pyrolysis reaction;
wherein the methane oxidative coupling reaction occurs in an oxidative conversion zone in a reactor and the pyrolyzing occurs in a pyrolysis zone in the same reactor.

13. The process of claim 1, further comprising introducing a hydrocarbon stream to the pyrolysis zone.

14. The process of claim 13, wherein the hydrocarbon stream comprises naphtha.

15. The process of claim 13, wherein the hydrocarbon stream comprises ethane.

16. The process of claim 1, wherein the coupling reaction occurs at a temperature of 750 to 900° C.

17. The process of claim 16, wherein the reaction occurs at a temperature of 790 to 810° C.

18. The process of claim 1, further comprising separating a purified ethylene from the second product stream.

19. A process for producing ethylene comprising:
separating natural gas into a methane stream and an ethane stream;
introducing the methane stream comprising methane, oxygen, and water to a methane coupling zone;
reacting the methane, the oxygen, and the water in the methane coupling zone via a methane oxidative coupling reaction to produce a first product stream;
introducing the first product stream and the ethane stream to a pyrolysis zone;
pyrolyzing ethane in the first product stream in the pyrolysis zone to produce a second product stream comprising greater than or equal to 10 vol % ethylene based on the total volume of the second product stream; and
separating a recycle ethane stream from the second product stream and introducing the recycle ethane stream to the pyrolysis zone;
wherein a heat from the methane coupling reaction is used in the pyrolysis reaction.

20. The process of claim 19, wherein the methane oxidative coupling reaction occurs in an oxidative conversion zone in a reactor and the pyrolyzing occurs in a pyrolysis zone in the same reactor.
